(19) United States
(12) Patent Application Publication
(10) Pub. No.: US 2019/0169090 A1
(43) Pub. Date: Jun. 6, 2019

(54) Oxidative Coupling of Methane Process with Enhanced Selectivity to C2+ Hydrocarbons by Addition of H2O in the Feed

(71) Applicant: Sabic Global Technologies, B.V., BERGEN OP ZOOM (NL)

(72) Inventors: Vidya Sagar Reddy SARSANI, Sugar Land, TX (US); Istvan LENGYEL, Sugar Land, TX (US); David WEST, Sugar Land, TX (US)

(73) Assignee: Sabic Global Teehnologies, B.V., BERGEN OP ZOOM (NL)

(21) Appl. No.: 16/322,561

(22) PCT Filed: Jul. 17, 2017
(86) PCT No.: PCT/US17/42376

§ 371 Date: Feb. 1, 2019

Related U.S. Application Data
(60) Provisional application No. 62/369,380, filed on Aug. 1, 2016.

(51) Int. Cl. C07C 2/84

(52) U.S. Cl. CPC C07C 2/84; C07C 11/04

(57) ABSTRACT

A process for producing olefins comprising introducing to a reactor a reactant mixture comprising methane, oxygen, and water, wherein the reactor comprises a catalyst, and wherein water is present in the reactant mixture from 0.5 mol % to 20 mol %; allowing the reactant mixture to contact the catalyst and react via an OCM reaction to form a product mixture comprising C₂₊ hydrocarbons, unreacted methane, and byproducts; wherein C₂₊ hydrocarbons comprise olefins and paraffins; and wherein the process is characterized by a productivity, a C₂₊ selectivity, or both that is increased when compared to a productivity, a C₂₊ selectivity, or both, respectively, of an otherwise similar process conducted (i) with a reactant mixture comprising methane and oxygen and (ii) without water present in the reactant mixture from 0.5 mol % to 20 mol %; recovering the product mixture from the reactor; recovering C₂₊ hydrocarbons from the product mixture; and recovering olefins from C₂₊ hydrocarbons.

### CROSS REFERENCE TO RELATED APPLICATIONS

This application is a filing under 35 U.S.C. 371 of International Application No. PCT/US2017/042376 filed Jul. 17, 2017, entitled, “Oxidative Coupling of Methane Process with Enhanced Selectivity to C2+ Hydrocarbons by Addition of H2O in the Feed,” which claims the benefit of U.S. Provisional Application No. 62/369,380 filed Aug. 1, 2016, entitled “Oxidative Coupling of Methane Process with Enhanced Selectivity to C2+ Hydrocarbons by Addition of H2O in the Feed,” which are incorporated by referenced herein in their entirety.

### TECHNICAL FIELD

The present disclosure relates to methods of producing olefins, more specifically methods of producing olefins by oxidative coupling of methane.

### BACKGROUND

Hydrocarbons, and specifically olefins such as ethylene, are typically building blocks used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Oxidative coupling of the methane (OCM) has been the target of intense scientific and commercial interest for more than thirty years due to the tremendous potential of such technology to reduce costs, energy, and environmental emissions in the production of ethylene (C₂H₄). As an overall reaction, in the OCM, CH₄ and O₂ react exothermically over a catalyst to form C₂H₄, water (H₂O) and heat.

Ethylene can be produced by OCM as represented by Equations (I) and (II):

2CH₄+O₂→C₂H₄+2H₂O ΔH=−67 kcal/mol (I)

2CH₄+ 1/20₂→C₂H₆+H₂O ΔH=−42 kcal/mol (II)

Oxidative conversion of methane to ethylene is exothermic. Excess heat produced from these reactions (Equations (I) and (II)) can push conversion of methane to carbon monoxide and carbon dioxide rather than the desired C₂ hydrocarbon product (e.g., ethylene):

CH₄+1.5O₂→CO+2H₂O ΔH=−124 kcal/mol (III)

CH₄+2O₂→CO₂+2H₂O ΔH=−192 kcal/mol (IV)

The excess heat from the reactions in Equations (III) and (IV) further exasperate this situation, thereby substantially reducing the selectivity of ethylene production when compared with carbon monoxide and carbon dioxide production.

Additionally, while the overall OCM is exothermic, catalysts are used to overcome the endothermic nature of the C—H bond breakage. The endothermic nature of the bond breakage is due to the chemical stability of methane, which is a chemically stable molecule due to the presence of its four strong tetrahedral C—H bonds (435 kJ/mol). When catalysts are used in the OCM, the exothermic reaction can lead to a large increase in catalyst bed temperature and uncontrolled heat excursions that can lead to catalyst deactivation and a further decrease in ethylene selectivity. Furthermore, the produced ethylene is highly reactive and can form unwanted and thermodynamically favored deep oxidation products.

Generally, in the OCM, CH₄ is first oxidatively converted into ethane (C₂H₆), and then into C₂H₄. CH₄ is activated heterogeneously on a catalyst surface, forming methyl free radicals (e.g., CH₃), which then couple in a gas phase to form C₂H₆. C₂H₆ subsequently undergoes dehydrogenation to form C₂H₄. An overall yield of desired C₂ hydrocarbons is reduced by non-selective reactions of methyl radicals with oxygen on the catalyst surface and/or in the gas phase, which produce (undesirable) carbon monoxide and carbon dioxide. Some of the best reported OCM outcomes encompass a ˜20% conversion of methane and ˜80% selectivity to desired C₂ hydrocarbons. Thus, there is an ongoing need for the development of OCM processes.

### BRIEF SUMMARY

Disclosed herein is a process for producing olefins comprising (a) introducing a reactant mixture to a reactor, wherein the reactant mixture comprises methane, oxygen, and water, wherein the reactor comprises a catalyst, and wherein the water is present in the reactant mixture in an amount of from about 0.5 mol % to about 20 mol %, (b) allowing at least a portion of the reactant mixture to contact the catalyst and react via an oxidative coupling of methane (OCM) reaction to form a product mixture; wherein the product mixture comprises C₂₊ hydrocarbons, unreacted methane, and byproducts; wherein the C₂₊ hydrocarbons comprise olefins and paraffins; and wherein the process is characterized by a productivity, a C₂₊ selectivity, or both that is increased when compared to a productivity, a C₂₊ selectivity, or both, respectively, of an otherwise similar process conducted (i) with a reactant mixture comprising methane and oxygen and (ii) without the water present in the reactant mixture in an amount of from about 0.5 mol % to about 20 mol %, (c) recovering at least a portion of the product mixture from the reactor, (d) recovering at least a portion of the C₂₊ hydrocarbons from the product mixture, and (e) recovering at least a portion of the olefins from the C₂₊ hydrocarbons.

Also disclosed herein is a process for producing olefins comprising (a) introducing a first reactant mixture to a first reactor, wherein the first reactant mixture comprises methane, oxygen, and water, wherein the first reactor comprises a first catalyst, and wherein the water is present in the first reactant mixture in an amount of from about 0.5 mol % to about 20 mol %, (b) allowing at least a portion of the first reactant mixture to contact the first catalyst and react via an oxidative coupling of methane (OCM) reaction to form a first product mixture; wherein the first product mixture comprises C₂₊ hydrocarbons, unreacted methane, and byproducts; wherein the C₂₊ hydrocarbons comprise olefins and paraffins; and wherein the byproducts comprise carbon monoxide, carbon dioxide, water, and hydrogen, (c) recovering at least a portion of the first product mixture from the first reactor, (d) removing a portion of the water from the first product mixture to produce a first intermediate mixture, (e) introducing a second reactant mixture to a second reactor comprising a second catalyst, wherein the second reactant mixture comprises at least a portion of the first intermediate mixture and oxygen, wherein the second reactant mixture comprises water in an amount of from about 0.5 mol % to about 20 mol %, and wherein the first catalyst and the second catalyst are the same or different, (f) allowing at least a portion of the second reactant mixture to contact the second catalyst and react via an OCM reaction to form a second product mixture; wherein the second product mixture comprises C₂₊ hydrocarbons, unreacted methane, and byproducts; wherein an amount of unreacted methane in the second product mixture is less than an amount of unreacted methane in the first product mixture; and wherein an amount of olefins in the second product mixture is greater than an amount of olefins in the first product mixture, (g) recovering at least a portion of the second product mixture from the second reactor, (h) optionally removing a portion of the water from the second product mixture to produce a second intermediate mixture, and (h) recovering at least a portion of the olefins from the second product mixture and/or the second intermediate mixture.

Further disclosed herein is a system for producing olefins comprising (a) a first oxidative coupling of methane (OCM) stage comprising (i) a first adiabatic reactor comprising a first catalyst, wherein the first adiabatic reactor is configured to receive a first reactant mixture comprising methane, oxygen, and water, wherein the water is present in the first reactant mixture in an amount of from about 0.5 mol % to about 20 mol %; and to produce a first product mixture; wherein the first product mixture comprises C₂₊ hydrocarbons, unreacted methane, and byproducts; wherein the C₂₊ hydrocarbons comprise olefins and paraffins; and wherein the byproducts comprise carbon monoxide, carbon dioxide, water, and hydrogen, and (ii) a first separating unit configured to receive at least a portion of the first product mixture and to produce a first intermediate mixture, wherein an amount of water in the first intermediate mixture is less than an amount of water in the first product mixture, (b) a second OCM stage comprising (iii) a second adiabatic reactor comprising a second catalyst, wherein the second adiabatic reactor is configured to receive a second reactant mixture comprising at least a portion of the first intermediate mixture and oxygen, wherein the water is present in the second reactant mixture in an amount of from about 0.5 mol % to about 20 mol %; and to produce a second product mixture; wherein the second product mixture comprises C₂₊ hydrocarbons, unreacted methane, and byproducts; wherein an amount of unreacted methane in the second product mixture is less than an amount of unreacted methane in the first product mixture; and wherein an amount of olefins in the second product mixture is greater than an amount of olefins in the first product mixture, and (iv) an optional second separating unit configured to receive at least a portion of the second product mixture and to produce a second intermediate mixture, wherein an amount of water in the second intermediate mixture is less than an amount of water in the second product mixture, and (c) a third separating unit configured to receive at least a portion of the second product mixture and/or the second intermediate mixture and to produce olefins.

### DETAILED DESCRIPTION

Disclosed herein are methods for producing olefins comprising (a) introducing a reactant mixture to a reactor, wherein the reactant mixture comprises methane, oxygen, and water, wherein the reactor comprises a catalyst, and wherein the water is present in the reactant mixture in an amount of from about 0.5 mol % to about 20 mol %; (b) allowing at least a portion of the reactant mixture to contact the catalyst and react via an oxidative coupling of methane (OCM) reaction to form a product mixture; wherein the product mixture comprises C₂₊ hydrocarbons, unreacted methane, and byproducts; wherein the C₂₊ hydrocarbons comprise olefins and paraffins; and wherein the process is characterized by a productivity, a C₂₊ selectivity, or both that is increased when compared to a productivity, a C₂₊ selectivity, or both, respectively, of an otherwise similar process conducted (i) with a reactant mixture comprising methane and oxygen and (ii) without the water present in the reactant mixture in an amount of from about 0.5 mol % to about 20 mol %; (c) recovering at least a portion of the product mixture from the reactor; (d) recovering at least a portion of the C₂₊ hydrocarbons from the product mixture; and (e) recovering at least a portion of the olefins from the C₂₊ hydrocarbons. In some aspects, the reactor can be an adiabatic reactor. Producing olefins can be a multi-stage process, wherein a first stage comprises steps (a) through (c), and wherein the multi-stage process further comprises one or more additional stages downstream of the first stage, as necessary to achieve a target methane conversion and/or a target C₂₊ selectivity for the overall multi-stage process.

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an aspect,” “another aspect,” “other aspects,” “some aspects,” and so forth, means that a particular element (e.g., feature, structure, property, and/or characteristic) described in connection with the aspect is included in at least an aspect described herein, and may or may not be present in other aspects. In addition, it is to be understood that the described element(s) can be combined in any suitable manner in the various aspects.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through the carbon of the carbonyl group.

A process for producing olefins can comprise introducing a reactant mixture to a reactor, wherein the reactant mixture comprises methane, oxygen, and water, wherein the reactor comprises a catalyst; and allowing at least a portion of the reactant mixture to contact the catalyst and react via an oxidative coupling of methane (OCM) reaction to form a product mixture; wherein the product mixture comprises C₂₊ hydrocarbons, unreacted methane, and byproducts; and wherein the C₂₊ hydrocarbons comprise olefins and paraffins. In an aspect, the process for producing olefins as disclosed herein can be a single-stage process, i.e., the process for producing olefins can employ a single reactor (e.g., single OCM reactor). For purposes of the disclosure herein a stage of a process, whether part of a single-stage process or part of a multi-stage process, can be defined as a single pass conversion through a single catalyst bed. While the current disclosure will be discussed in detail in the context of a single stage comprising a single reactor comprising a single catalyst bed, it should be understood that any suitable stage/reactor/catalyst bed configurations can be used. For example, two or more stages of a multi-stage process can be housed in one or more reactors. As will be appreciated by one of skill in the art, and with the help of this disclosure, multiple stages can be housed within a single reaction vessel, for example a vessel comprising two or more catalyst beds in series. Further, as will be appreciated by one of skill in the art, and with the help of this disclosure, multiple vessels can be part of a single stage, for example two or more vessels in parallel, wherein a reactant mixture is distributed between and introduced to the two or more vessels in parallel.

In an aspect, the reactor (e.g., OCM reactor) can be an adiabatic reactor. The OCM reactors can be fixed bed reactors, such as axial flow reactors, or radial flow reactors. As will be appreciated by one of skill in the art, and with the help of this disclosure, certain fixed bed reactors, such as radial flow reactors, can decrease a reactor pressure drop, which may in turn increase a desired selectivity.

In an aspect, the OCM reaction can be conducted in the reactor at a temperature of from about 750° C. to about 1,000° C., alternatively from about 775° C. to about 975° C., or alternatively from about 800° C. to about 950° C.

The reactor can comprise a catalyst (e.g., an OCM catalyst). The catalyst can comprise basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; and the like; or combinations thereof.

In an aspect, the catalyst comprises one or more oxides. Nonlimiting examples of the one or more oxides suitable for use in the present disclosure include CeO₂, La₂O₃—CeO₂, Ca/CeO₂, Mn/Na₂WO₄, Li₂O, Na₂O, Cs₂O, WO₃, Mn₃O₄, CaO, MgO, SrO, BaO, CaO—MgO, CaO—BaO, Li/MgO, MnO, W₂O₃, SnO₂, Yb₂O₃, Sm₂O₃, MnO—W₂O₃, MnO—W₂O₃—Na₂O, MnO—W₂O₃—Li₂O, SrO/La₂O₃, Ce₂O₃, La/MgO, La₂O₃—CeO₂—Na₂O, La₂O₃—CeO₂—CaO, Na₂O—MnO—WO₃—La₂O₃, La₂O₃—CeO₂—MnO—WO₃—SrO, Na—Mn—La₂O₃/Al₂O₃, Na—Mn—O/SiO₂, Na₂WO₄—Mn/SiO₂, Na₂WO₄—Mn—O/SiO₂, Na/Mn/O, Na₂WO₄, Mn₂O₃/Na₂WO₄, Mn₃O₄/Na₂WO₄, MnWO₄/Na₂WO₄, MnWO₄/Na₂WO₄, Mn/WO₄, Na₂WO₄/Mn, Sr/Mn—Na₂WO₄, and the like, or combinations thereof.

In an aspect, the catalysts suitable for use in the present disclosure can be supported catalysts and/or unsupported catalysts. In some aspects, the supported catalysts can comprise a support, wherein the support can be catalytically active (e.g., the support can catalyze an OCM reaction). For example, the catalytically active support can comprise a metal oxide support, such as MgO. In other aspects, the supported catalysts can comprise a support, wherein the support can be catalytically inactive (e.g., the support cannot catalyze an OCM reaction), such as SiO₂. In yet other aspects, the supported catalysts can comprise a catalytically active support and a catalytically inactive support.

In some aspects, the support comprises an inorganic oxide, alpha, beta or theta alumina (Al₂O₃), activated Al₂O₃, silicon dioxide (SiO₂), titanium dioxide (TiO₂), magnesium oxide (MgO), calcium oxide (CaO), strontium oxide (SrO), zirconium oxide (ZrO₂), zinc oxide (ZnO), lithium aluminum oxide (LiAlO₂), magnesium aluminum oxide (MgAlO₄), manganese oxides (MnO, MnO₂, Mn₃O₄), lanthanum oxide (La₂O₃), activated carbon, silica gel, zeolites, activated clays, silicon carbide (SiC), diatomaceous earth, magnesia, aluminosilicates, calcium aluminate, carbonates, MgCO₃, CaCO₃, SrCO₃, BaCO₃, Y₂(CO₃)₃, La₂(CO₃)₃, and the like, or combinations thereof. In an aspect, the support can comprise MgO, Al₂O₃, SiO₂, ZrO₂, and the like, or combinations thereof.

The reactant mixture can comprise a hydrocarbon or mixtures of hydrocarbons, and oxygen. In some aspects, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons, such as diesel fuel, jet fuel, gasoline, tars, kerosene, etc.), oxygenated hydrocarbons, biodiesel, alcohols, dimethyl ether, and the like, or combinations thereof. In an aspect, the reactant mixture can comprise CH₄, O₂, and water.

The O₂ used in the reactant mixture can be oxygen gas (which may be obtained via a membrane separation process), technical oxygen (which may contain some air), air, oxygen enriched air, and the like, or combinations thereof.

The reactant mixture can further comprise a diluent. For purposes of the disclosure herein the term “diluent” excludes water or steam. The diluent is inert with respect to the OCM reaction, e.g., the diluent does not participate in the OCM reaction. In an aspect, the diluent can comprise, nitrogen, inert gases (e.g., argon), and the like, or combinations thereof. In an aspect, the diluent can be present in the reactant mixture in an amount of from about 0.5% to about 80%, alternatively from about 5% to about 50%, or alternatively from about 10% to about 30%, based on the total volume of the reactant mixture.

In an aspect, the reactant mixture comprises water or steam. As will be appreciated by one of skill in the art, and with the help of this disclosure, the water can be present in the reactant mixture in the form of steam, depending on the temperature and pressure of the reactant mixture. In an aspect, water can be present in the reactant mixture in an amount of from about 0.5 mol % to about 20 mol %, alternatively from about 2.5 mol % to about 17.5 mol %, or alternatively from about 5 mol % to about 15 mol %. For purposes of the disclosure herein, the amount of water present in the reactant mixture refers to a cumulative amount of water in the reactant mixture, such as for example an amount of water owing to water introduced as water or steam to the reactant mixture, water present in a methane or natural gas feed, water present in an optional diluent, steam introduced to a reactor, etc. The water can comprise tap water, process water, etc.

In an aspect, the reactant mixture can comprise steam, e.g., the water is in the form of steam (in the reactor) at the OCM reaction conditions present in the reactor.

In an aspect, a process for producing olefins can comprise recovering at least a portion of the product mixture from the reactor. The product mixture can comprise C₂₊ hydrocarbons (e.g., olefins and paraffins), unreacted methane, and byproducts, such as water, carbon monoxide (CO), carbon dioxide (CO₂), and hydrogen. The process can comprise recovering at least a portion of the C₂₊ hydrocarbons from the product mixture.

In an aspect, the process for producing olefins as disclosed herein can be characterized by a productivity that is increased when compared to a productivity of an otherwise similar process conducted (i) with a reactant mixture comprising methane and oxygen and (ii) without the water present in the reactant mixture in an amount of from about 0.5 mol % to about 20 mol %. For purposes of the disclosure herein, the term “productivity” refers to conversion rate of a reagent or reactant, such as methane, per unit volume of the catalyst bed. For purposes of the disclosure herein, the conversion of a reagent is a % conversion based on moles converted. For example, the methane conversion can be calculated by using equation (1):

$${CH}_{4} conversion = \frac{{C}_{{CH}_{4}}^{in} - {C}_{{CH}_{4}}^{o ut}}{{C}_{{CH}_{4}}^{in}} \times 100 \% \qquad ( 1 )$$

wherein C_{CH}_{₄}ⁱⁿ=number of moles of C from CH₄ that entered the reactor as part of the reactant mixture; and C_{CH}_{₄}^{out}=number of moles of C from CH₄ that was recovered from the reactor as part of the product mixture.

In an aspect, the process for producing olefins as disclosed herein can be characterized by a productivity that is increased by equal to or greater than about 1%, alternatively equal to or greater than about 2%, alternatively equal to or greater than about 5%, or alternatively equal to or greater than about 10%, when compared to a productivity of an otherwise similar process conducted (i) with a reactant mixture comprising methane and oxygen and (ii) without the water present in the reactant mixture in an amount of from about 0.5 mol % to about 20 mol %. For purposes of the disclosure herein, a quantity y that is increased by x % to give yl can be calculated as follows: yl=[y+(y·x/100)]. For example, if a productivity is 15%, when this productivity is increased by 10% the resulting productivity is 16.5%.

In an aspect, an oxygen conversion can be from about 90% to about 100%, alternatively from about 95% to 99.99%, or alternatively from about 98% to 99.9%. For example, the oxygen conversion can be calculated by using equation (2):

$$Oxygen conversion = \frac{{Moles}_{{O}_{2}}^{in} - {Moles}_{{O}_{2}}^{out}}{{Moles}_{{O}_{2}}^{in}} \times 100 \% \qquad ( 2 )$$

wherein Moles_{O}_{₂}ⁱⁿ=number of moles of oxygen that was introduced to the reactor; and Moles_{O}_{₂}^{out}=number of moles of oxygen that was recovered from the reactor.

In an aspect, the process for producing olefins as disclosed herein can be characterized by a C₂₊ selectivity that is increased when compared to a C₂₊ selectivity of an otherwise similar process conducted (i) with a reactant mixture comprising methane and oxygen and (ii) without the water present in the reactant mixture in an amount of from about 0.5 mol % to about 20 mol %.

Generally, a selectivity to a desired product or products refers to how much desired product was formed divided by the total products formed, both desired and undesired. For purposes of the disclosure herein, the selectivity to a desired product is a % selectivity based on moles converted into the desired product. Further, for purposes of the disclosure herein, a Cₓ selectivity (e.g., C₂₊ selectivity, C₂ selectivity, etc.) can be calculated by dividing a number of moles of carbon (C) from CH₄ that were converted into the desired product (e.g., C_{C2H4}, C_{C2H6}, etc.) by the total number of moles of C from CH₄ that were converted (e.g., C_{C2H4}, C_{C2H6}, C_{C2H2}, C_{C3H6}, C_{C3H8}, C_{C4s}, C_{CO2}, C_{CO}, etc.). C_{C2H4}=number of moles of C from CH₄ that were converted into C₂H₄; C_{C2H6}=number of moles of C from CH₄ that were converted into C₂H₆; C_{c212}=number of moles of C from CH₄ that were converted into C₂H₂; C_{C3H6}=number of moles of C from CH₄ that were converted into C₃H₆; C_{C3H8}=number of moles of C from CH₄ that were converted into C₃H₈; C_{C4s}=number of moles of C from CH₄ that were converted into C₄ hydrocarbons (C₄s); C_{CO2}=number of moles of C from CH₄ that were converted into CO₂; C_{co}=number of moles of C from CH₄ that were converted into CO; etc.

A C₂₊ selectivity (e.g., selectivity to C₂₊ hydrocarbons) refers to how much C₂H₄, C₃H₆, C₂H₂, C₂H₆, C₃H₈, and C₄s were formed divided by the total products formed, including C₂H₄, C₃H₆, C₂H₂, C₂H₆, C₃H₈, C₄s, CO₂ and CO. For example, the C₂₊ selectivity can be calculated by using equation (3):

$${C}_{2 +} selectivity = \frac{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4 s}}\end{matrix}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4 s}} + {C}_{{CO}_{2}} + {C}_{CO}\end{matrix}} \times 100 \% \qquad ( 3 )$$

As will be appreciated by one of skill in the art, and with the help of this disclosure, if a specific product and/or hydrocarbon product having x number of carbon atoms is not produced in a certain OCM reaction/process, then the corresponding C_{Cx} is 0, and the term is simply removed from selectivity calculations.

In an aspect, the process for producing olefins as disclosed herein can be characterized by a C₂₊ selectivity that is increased by equal to or greater than about 1%, alternatively equal to or greater than about 2%, alternatively equal to or greater than about 3%, alternatively equal to or greater than about 4%, or alternatively equal to or greater than about 5%, when compared to a C₂₊ selectivity of an otherwise similar process conducted (i) with a reactant mixture comprising methane and oxygen and (ii) without the water present in the reactant mixture in an amount of from about 0.5 mol % to about 20 mol %. The process for producing olefins can comprise recovering at least a portion of the olefins from the C₂₊ hydrocarbons.

In an aspect, a process for producing olefins can comprise cooling the product mixture, for example in a heat exchanger. In some aspects, the heat exchanger can generate steam by heating water with the heat captured from the product mixture, wherein such steam can be further used in the reactant mixture. In other aspects, the heat exchanger can heat the reactant mixture comprising water, thereby generating steam within the reactant mixture and providing at least a portion of the heat necessary to initiate the OCM reaction.

A process for producing olefins can comprise multiple stages (e.g., as part of a multi-stage process), wherein each individual stage can comprise an OCM reactor, wherein each individual stage can be repeated as necessary to achieve a target productivity for the overall multi-stage process. A multi-stage process generally comprises a plurality of individual stages, wherein each individual stage comprises a single pass conversion through a single catalyst bed. While the current disclosure will be discussed in detail in the context of a multi-stage process comprising 2 stages, it should be understood that any suitable number of stages can be used, such as for example, 2 stages, 3 stages, 4 stages, 5 stages, 6 stages, 7 stages, 8 stages, 9 stages, 10 stages, or more stages. For purposes of the disclosure herein, all descriptions related to the single-stage process (such as descriptions of reactor, catalyst, reactant mixture, product mixture, heat exchanger, etc.) can be applied to the corresponding components of any stage of a multi-stage process (such as descriptions of reactors (e.g., first reactor, second reactor), catalysts (first catalyst, second catalyst), reactant mixtures (e.g., first reactant mixture, second reactant mixture), product mixtures (e.g., first product mixture, second product mixture), heat exchangers (e.g., first heat exchanger, second heat exchanger), etc., respectively), unless otherwise specified herein.

In an aspect, a process for producing olefins can comprise a first stage (e.g., a first OCM stage), wherein the first stage comprises (a) introducing a first reactant mixture to a first reactor, wherein the first reactant mixture comprises methane, oxygen, and water, wherein the first reactor comprises a first catalyst, and wherein the water is present in the first reactant mixture in an amount of from about 0.5 mol % to about 20 mol %; (b) allowing at least a portion of the first reactant mixture to contact the first catalyst and react via an OCM reaction to form a first product mixture; wherein the first product mixture comprises C₂₊ hydrocarbons, unreacted methane, and byproducts; wherein the C₂₊ hydrocarbons comprise olefins and paraffins; and wherein the byproducts comprise carbon monoxide, carbon dioxide, water, and hydrogen; (c) recovering at least a portion of the first product mixture from the first reactor; and (d) removing a portion of the water from the first product mixture to produce a first intermediate mixture.

In an aspect, the step of removing a portion of the water from the first product mixture to produce a first intermediate mixture can further comprise cooling the first product mixture, for example in a first heat exchanger. The first heat exchanger can generate steam by heating water with the heat captured from the first product mixture, wherein such steam can be further used in the first reactant mixture.

In an aspect, a process for producing olefins can be a multi-stage process, wherein the multi-stage process further comprises one or more additional OCM stages downstream of the first stage, as necessary to achieve a target productivity and/or a target C₂₊ selectivity for the overall multi-stage process. In an aspect, the multi-stage process can have from 2 to about 5 stages, alternatively from 3 to about 5 stages, or alternatively from 3 to 4 stages. Each additional OCM stage can comprise (i) introducing a reactant mixture to a reactor, wherein the reactant mixture comprises methane, oxygen, and water, wherein the reactor comprises a catalyst, and wherein the water is present in the reactant mixture in an amount of from about 0.5 mol % to about 20 mol %; (ii) allowing at least a portion of the reactant mixture to contact the catalyst and react via an OCM reaction to form a product mixture, wherein the product mixture comprises C₂₊ hydrocarbons, unreacted methane, and byproducts, and wherein the C₂₊ hydrocarbons comprise olefins and paraffins; and (iii) recovering at least a portion of the product mixture from the reactor.

In some aspects, the reactant mixture can comprise a portion of an upstream product mixture recovered from an upstream reactor. In such aspects, the process can further comprise (i) removing a portion of water from the upstream product mixture to produce an intermediate mixture; and (ii) contacting at least a portion of the intermediate mixture with oxygen to produce the reactant mixture, wherein the reactant mixture comprises water in an amount of from about 0.5 mol % to about 20 mol %. As will be appreciated by one of skill in the art, and with the help of this disclosure, water is a product of the OCM reaction, and as such, a product mixture will have a greater water content when compared to a reactant mixture for the same reactor. Further, as will be appreciated by one of skill in the art, and with the help of this disclosure, in multi-stage processes, in order to maintain a water amount of from about 0.5 mol % to about 20 mol %, an upstream product mixture has to be subjected to a water removal step, i.e., an upstream product mixture has to be subjected to a step of removing the water that was produced in the upstream OCM reaction.

In an aspect, a portion of the water can be removed from the upstream product mixture, to yield an intermediate mixture. In an aspect, the upstream product mixture can be introduced to a compressor, and then to a water quench vessel (e.g., a separating unit). Generally, compressing a gas that contains water from a first pressure to a second pressure (wherein the second pressure is greater than the first pressure) will lead to the water condensing at the second pressure at an increased temperature as compared to a temperature where water of an otherwise similar gas condenses at the first pressure. In an aspect, the compressed upstream product mixture can be further cooled in a cooling tower (e.g., heat exchanger, first heat exchanger) or in the water quench vessel to promote water condensation and removal.

In other aspects, the reactant mixture can further comprise at least a portion of a downstream product mixture recovered from a downstream reactor (e.g., a recycle stream, such as for example recovered unreacted methane).

In an aspect, a process for producing olefins can comprise a first stage and a second stage, wherein the first stage comprises a first reactor, and wherein the second stage comprises a second reactor, and wherein the first reactor and the second reactor are in series, with the second reactor downstream of the first reactor.

In an aspect, a process for producing olefins can comprise (a) introducing a first reactant mixture to a first reactor, wherein the first reactant mixture comprises methane, oxygen, and water, wherein the first reactor comprises a first catalyst, and wherein the water is present in the first reactant mixture in an amount of from about 0.5 mol % to about 20 mol %; (b) allowing at least a portion of the first reactant mixture to contact the first catalyst and react via an OCM reaction to form a first product mixture; wherein the first product mixture comprises C₂₊ hydrocarbons, unreacted methane, and byproducts; wherein the C₂₊ hydrocarbons comprise olefins and paraffins; and wherein the byproducts comprise carbon monoxide, carbon dioxide, water, and hydrogen; (c) recovering at least a portion of the first product mixture from the first reactor; (d) removing a portion of the water from the first product mixture to produce a first intermediate mixture; (e) introducing a second reactant mixture to a second reactor comprising a second catalyst, wherein the second reactant mixture comprises at least a portion of the first intermediate mixture and oxygen, wherein the second reactant mixture comprises water in an amount of from about 0.5 mol % to about 20 mol %, and wherein the first catalyst and the second catalyst are the same or different; (f) allowing at least a portion of the second reactant mixture to contact the second catalyst and react via an OCM reaction to form a second product mixture; wherein the second product mixture comprises C₂₊ hydrocarbons, unreacted methane, and byproducts; wherein an amount of unreacted methane in the second product mixture is less than an amount of unreacted methane in the first product mixture, with the proviso that no fresh or supplemental methane is added to the second stage to desirably produce an increase in a methane concentration; and wherein an amount of olefins in the second product mixture is greater than an amount of olefins in the first product mixture, with the proviso that no olefins are separated or recovered from the first product mixture to desirably produce a decrease in an olefin concentration; (g) recovering at least a portion of the second product mixture from the second reactor; (h) optionally removing a portion of the water from the second product mixture to produce a second intermediate mixture; and (h) recovering at least a portion of the olefins from the second product mixture and/or the second intermediate mixture. In such aspect, producing olefins can be a multi-stage process, wherein a first stage comprises steps (a) through (d), wherein a second stage comprises steps (e) through (h), and wherein the multi-stage process further comprises one or more additional stages downstream of the first stage and/or the second stage, as necessary to achieve a target productivity and/or a target C₂₊ selectivity for the overall multi-stage process. For purposes of the disclosure herein, all descriptions related to the single-stage process and/or the first stage (such as descriptions of reactor (e.g., first reactor), catalyst (e.g., first catalyst), reactant mixture (e.g., first reactant mixture), product mixture (e.g., first product mixture), intermediate mixture (e.g., first intermediate mixture), heat exchanger (e.g., first heat exchanger) etc.) can be applied to the corresponding components of the second (such as descriptions of second reactor, second catalyst, second reactant mixture, second product mixture, second intermediate mixture, second heat exchanger, etc., respectively), unless otherwise specified herein.

As will be appreciated by one of skill in the art, and with the help of this disclosure, the methane reacting in the second stage in the second reactor is primarily methane that was introduced to the first reactor, that didn't react in the first reactor, and that was subsequently recovered as unreacted methane (as part of the first product mixture), with the proviso that no fresh or supplemental methane was added to the second stage to desirably produce an increase in a methane concentration.

In an aspect, each additional stage can comprise (i) introducing a reactant mixture to a reactor, wherein the reactant mixture comprises methane, oxygen, and water, wherein the reactor comprises a catalyst, and wherein the water is present in the reactant mixture in an amount of from about 0.5 mol % to about 20 mol %; (ii) allowing at least a portion of the reactant mixture to contact the catalyst and react via an OCM reaction to form a product mixture, wherein the product mixture comprises C₂₊ hydrocarbons, unreacted methane, and byproducts, wherein the C₂₊ hydrocarbons comprise olefins and paraffins, and wherein the byproducts comprise carbon monoxide, carbon dioxide, water, and hydrogen; and (iii) recovering at least a portion of the product mixture from the reactor; and (iv) optionally removing a portion of the water from the product mixture to produce an intermediate mixture. In some aspects, the reactant mixture comprises at least a portion of an upstream intermediate mixture recovered from an upstream reactor.

In an aspect, the multi-stage process can be characterized by an overall productivity, an overall C₂₊ selectivity, or both that is increased when compared to an overall productivity, an overall C₂₊ selectivity, or both, respectively, of an otherwise similar process conducted with (i) a first reactant mixture comprising methane and oxygen without the water present in the first reactant mixture in an amount of from about 0.5 mol % to about 20 mol %, and (ii) a second reactant mixture comprising methane and oxygen without the water present in the second reactant mixture in an amount of from about 0.5 mol % to about 20 mol %.

In an aspect, the overall productivity for a multi-stage process can be increased by equal to or greater than about 2%, alternatively equal to or greater than about 5%, alternatively equal to or greater than about 10%, or alternatively equal to or greater than about 15%, when compared to an overall productivity of an otherwise similar multi-stage process conducted with (i) a first reactant mixture comprising methane and oxygen without the water present in the first reactant mixture in an amount of from about 0.5 mol % to about 20 mol %, and (ii) a second reactant mixture comprising methane and oxygen without the water present in the second reactant mixture in an amount of from about 0.5 mol % to about 20 mol %. For purposes of the disclosure herein, the overall productivity refers to the overall conversion rate of a reagent or reactant, such as methane, per unit volume of the catalyst bed. For example, the overall methane conversion in a multi-stage process can be calculated by using equation (4):

$$Methane multi \text{-} stage conversion = \frac{\begin{matrix}{Moles}_{{CH}_{4}}^{in multi \text{-} stage process} - \\ {Moles}_{{CH}_{4}}^{out multi \text{-} stage process}\end{matrix}}{{Moles}_{{CH}_{4}}^{in multi \text{-} stage process}} \times 100 \% \qquad ( 4 )$$

wherein Moles_{CH}_{₄}^{in multi-stage process}=number of moles of methane that was introduced to the multi-stage process; and Moles_{CH}_{₄}^{out multi stage process}=number of moles of methane that was recovered from the multi-stage process.

In an aspect, the overall C₂₊ selectivity for a multi-stage process can be increased by equal to or greater than about 1%, alternatively equal to or greater than about 2%, alternatively equal to or greater than about 5%, or alternatively equal to or greater than about 10%, when compared to an overall C₂₊ selectivity of an otherwise similar multi-stage process conducted with (i) a first reactant mixture comprising methane and oxygen without the water present in the first reactant mixture in an amount of from about 0.5 mol % to about 20 mol %, and (ii) a second reactant mixture comprising methane and oxygen without the water present in the second reactant mixture in an amount of from about 0.5 mol % to about 20 mol %. For example, the overall C₂₊ selectivity in a multi-stage process can be calculated by using equation (3), wherein the amount of products recovered in anywhere from the multi-stage process (e.g., C₂H₄, C₃H₆, C₂H₂, C₂H₆, C₃H₈, and C₄s) are divided by the amount of total products recovered anywhere from the multi-stage process (e.g., C₂H₄, C₃H₆, C₂H₂, C₂H₆, C₃H₈, C₄s, CO₂, and CO).

In an embodiment, a multi-stage process can comprise three or more stages, wherein the first stage can be referred to as an “initial stage,” the last stage can be referred to as a “terminal stage,” and one or more stage in between the first stage and the last stage can be referred to as “intermediate stages.” Selectivities and conversions can generally be calculated for multi-stage processes by using equations (3) and (4), via a mass balance of reactants introduced in any stage (e.g., initial stage, intermediate stage(s), terminal stage) and products and/or unreacted reagents recovered from any stage (e.g., initial stage, intermediate stage(s), terminal stage). For the multi-stage process disclosed herein, a methane conversion, for example, would account for methane introduced in the initial stage and for unconverted methane recovered from the terminal stage.

In an aspect, a process for producing ethylene can comprise (a) introducing a first reactant mixture to a first adiabatic reactor, wherein the first reactant mixture comprises methane, oxygen, and water, wherein the first adiabatic reactor comprises a first catalyst, wherein the first catalyst comprises one or more oxides, and wherein the water is present in the first reactant mixture in an amount of from about 5 mol % to about 15 mol %; (b) allowing at least a portion of the first reactant mixture to contact the first catalyst and react via an OCM reaction to form a first product mixture; wherein the first product mixture comprises C₂₊ hydrocarbons, unreacted methane, and byproducts; wherein the C₂₊ hydrocarbons comprise olefins and paraffins; wherein the olefins comprise ethylene; and wherein the byproducts comprise carbon monoxide, carbon dioxide, water, and hydrogen; (c) recovering at least a portion of the first product mixture from the first adiabatic reactor; (d) removing a portion of the water from the first product mixture to produce a first intermediate mixture; (e) introducing a second reactant mixture to a second adiabatic reactor comprising a second catalyst, wherein the second reactant mixture comprises at least a portion of the first intermediate mixture and oxygen, wherein the second reactant mixture comprises water in an amount of from about 5 mol % to about 15 mol %, wherein the second catalyst comprises one or more oxides, and wherein the first catalyst and the second catalyst are the same or different; (f) allowing at least a portion of the second reactant mixture to contact the second catalyst and react via an OCM reaction to form a second product mixture; wherein the second product mixture comprises C₂₊ hydrocarbons, unreacted methane, and byproducts; wherein an amount of unreacted methane in the second product mixture is less than an amount of unreacted methane in the first product mixture, with the proviso that no fresh or supplemental methane is added to the second adiabatic reactor to desirably produce an increase in a methane concentration; and wherein an amount of ethylene in the second product mixture is greater than an amount of ethylene in the first product mixture, with the proviso that no ethylene is separated or recovered from the first product mixture to desirably produce a decrease in an ethylene concentration; (g) recovering at least a portion of the second product mixture from the second adiabatic reactor; (h) optionally removing a portion of the water from the second product mixture to produce a second intermediate mixture; and (h) recovering at least a portion of the ethylene from the second product mixture and/or the second intermediate mixture. In such aspect, producing ethylene can be a multi-stage process, wherein a first stage comprises steps (a) through (d), wherein a second stage comprises steps (e) through (h), and wherein the multi-stage process further comprises one or more additional stages downstream of the first stage and/or the second stage, as necessary to achieve a target productivity and/or a target C₂ selectivity for the overall multi-stage process.

In an aspect, a system for producing ethylene can comprise (a) a first OCM stage comprising (i) a first adiabatic reactor comprising a first catalyst, wherein the first adiabatic reactor is configured to receive a first reactant mixture comprising methane, oxygen, and water, wherein the water is present in the first reactant mixture in an amount of from about 5 mol % to about 15 mol %; and to produce a first product mixture; wherein the first product mixture comprises C₂₊ hydrocarbons, unreacted methane, and byproducts; wherein the C₂₊ hydrocarbons comprise olefins and paraffins; wherein the olefins comprise ethylene; and wherein the byproducts comprise carbon monoxide, carbon dioxide, water, and hydrogen; and (ii) a first separating unit configured to receive at least a portion of the first product mixture and to produce a first intermediate mixture, wherein an amount of water in the first intermediate mixture is less than an amount of water in the first product mixture; (b) a second OCM stage comprising (iii) a second adiabatic reactor comprising a second catalyst, wherein the second adiabatic reactor is configured to receive a second reactant mixture comprising at least a portion of the first intermediate mixture and oxygen, wherein the water is present in the second reactant mixture in an amount of from about 5 mol % to about 15 mol %; and to produce a second product mixture; wherein the second product mixture comprises C₂₊ hydrocarbons, unreacted methane, and byproducts; wherein an amount of unreacted methane in the second product mixture is less than an amount of unreacted methane in the first product mixture, with the proviso that no fresh or supplemental methane is added to the second adiabatic reactor to desirably produce an increase in a methane concentration; and wherein an amount of ethylene in the second product mixture is greater than an amount of ethylene in the first product mixture, with the proviso that no ethylene is separated or recovered from the first product mixture to desirably produce a decrease in an ethylene concentration; and (iv) an optional second separating unit configured to receive at least a portion of the second product mixture and to produce a second intermediate mixture, wherein an amount of water in the second intermediate mixture is less than an amount of water in the second product mixture; and (c) a third separating unit configured to receive at least a portion of the second product mixture and/or the second intermediate mixture and to produce ethylene. The first separating unit and the optional second separating unit can comprise a heat exchanger, a cooling tower, a water quench vessel, or combinations thereof. In some aspects, the third separating unit is a distillation column, such as a cryogenic distillation column.

The system for producing ethylene can be characterized by an overall productivity, an overall C₂₊ selectivity, or both that is increased when compared to an overall productivity, an overall C₂₊ selectivity, or both, respectively, of an otherwise similar system having (i) a first reactant mixture comprising methane and oxygen without the water present in the first reactant mixture in an amount of from about 5 mol % to about 15 mol %, and (ii) a second reactant mixture comprising methane and oxygen without the water present in the second reactant mixture in an amount of from about 5 mol % to about 15 mol %.

In an aspect, a process for producing olefins as disclosed herein can advantageously display improvements in one or more method characteristics when compared to an otherwise similar process conducted (i) with a reactant mixture comprising methane and oxygen and (ii) without the water present in the reactant mixture in an amount of from about 0.5 mol % to about 20 mol %.

In an aspect, a process for producing olefins as disclosed herein can advantageously decrease deep oxidation reactions, thereby decreasing an amount of CO and/or CO₂ produced in the process.

In an aspect, a process for producing olefins as disclosed herein can advantageously provide for an increased overall productivity and/or an increased overall C₂₊ selectivity. Without wishing to be limited by theory, it is unexpected to note that the addition of water, which is a product of the OCM reaction, increases productivity and/or selectivity to desired products (e.g., C₂₊ selectivity, C₂ selectivity), as a reaction product is typically expected to drive the reaction equilibrium in the opposite direction (which would cause a decrease in productivity and/or selectivity to desired products). The increased productivity allows for advantageously processing more feedstock per the same volume of catalyst. Additional advantages of the processes for the production of olefins as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

Oxidative coupling of methane (OCM) reactions were conducted in the presence of a catalyst as follows. Methane, hydrogen and oxygen gases, along with an internal standard, an inert gas (neon) were fed to a quartz reactor with an internal diameter (I.D.) of 4 mm and were heated using a traditional clamshell furnace at a desired set point temperature. The reactor was first heated to a desired temperature under an inert gas flow and then a desired gas mixture was fed to the reactor. The OCM reaction was conducted both in the absence of water and in the presence of water, wherein water was present in the feed as steam in an amount of about 10 mol %.

Selectivities and conversions were calculated as outlined in equations (1)-(3), and the data are displayed in Table 1. The data in Table 1 were acquired in the presence of a Na₂WO₄—Mn—O/SiO₂, catalyst bed (100 mg loading), at a feed CH₄/O₂ molar ratio of 16:1; a furnace temperature of 750° C.; a residence time of 60 ms in the absence of water; and a residence time of 54 ms in the presence of water.

TABLE 1

| | | 10 mol % |
|---|---|---|
| | No Water | Water in the Feed |
| % CH4 Conversion | 11.3 | 13.0 |
| % O2 Conversion | 96.8 | 99.9 |
| ‘C’ Selectivities | | |
| C2= | 30.8 | 33.1 |
| C2= | 0.0 | 0.3 |
| C2 | 54.9 | 53.1 |
| C3= | 2.3 | 2.6 |
| C3 | 2.4 | 2.7 |
| C4= | 0.7 | 0.9 |
| % C2+ | 91.1 | 92.7 |
| % CO | 2.3 | 2.1 |
| % CO2 | 6.6 | 5.2 |

The data in Table 1 show that oxygen conversion was increased from about 97% in the absence of water to near complete oxygen conversion (99.9%) when water was added to the feed mixture, despite operating at a lower residence time. Further, the C₂₊ selectivity was enhanced by 1.6% when water was added to the feed mixture. Also, less CO and CO₂ were produced when water was present in the feed, indicating a reduction in undesirable deep oxidation reactions in the presence of water.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### Additional Disclosure

A first aspect, which is a process for producing olefins comprising (a) introducing a reactant mixture to a reactor, wherein the reactant mixture comprises methane, oxygen, and water, wherein the reactor comprises a catalyst, and wherein the water is present in the reactant mixture in an amount of from about 0.5 mol % to about 20 mol %; (b) allowing at least a portion of the reactant mixture to contact the catalyst and react via an oxidative coupling of methane (OCM) reaction to form a product mixture; wherein the product mixture comprises C₂₊ hydrocarbons, unreacted methane, and byproducts; wherein the C₂₊ hydrocarbons comprise olefins and paraffins; and wherein the process is characterized by a productivity, a C₂₊ selectivity, or both that is increased when compared to a productivity, a C₂₊ selectivity, or both, respectively, of an otherwise similar process conducted (i) with a reactant mixture comprising methane and oxygen and (ii) without the water present in the reactant mixture in an amount of from about 0.5 mol % to about 20 mol %; (c) recovering at least a portion of the product mixture from the reactor; (d) recovering at least a portion of the C₂₊ hydrocarbons from the product mixture; and (e) recovering at least a portion of the olefins from the C₂₊ hydrocarbons.

A second aspect, which is the process of the first aspect, wherein the reactor is an adiabatic reactor.

A third aspect, which is the process of any one of the first and the second aspects, wherein the process is characterized by a productivity that is increased by equal to or greater than about 1% when compared to a productivity of an otherwise similar process conducted (i) with a reactant mixture comprising methane and oxygen and (ii) without the water present in the reactant mixture in an amount of from about 0.5 mol % to about 20 mol %.

A fourth aspect, which is the process of any one of the first through the third aspects, wherein the process is characterized by a C₂₊ selectivity that is increased by equal to or greater than about 1% when compared to a C₂₊ selectivity of an otherwise similar process conducted (i) with a reactant mixture comprising methane and oxygen and (ii) without the water present in the reactant mixture in an amount of from about 0.5 mol % to about 20 mol %.

A fifth aspect, which is the process of any one of the first through the fourth aspects, wherein the process is characterized by a productivity that is increased by equal to or greater than about 2% when compared to a productivity of an otherwise similar process conducted (i) with a reactant mixture comprising methane and oxygen and (ii) without the water present in the reactant mixture in an amount of from about 0.5 mol % to about 20 mol %; and wherein the process is characterized by a C₂₊ selectivity that is increased by equal to or greater than about 1% when compared to a C₂₊ selectivity of an otherwise similar process conducted (i) with a reactant mixture comprising methane and oxygen and (ii) without the water present in the reactant mixture in an amount of from about 0.5 mol % to about 20 mol %.

A sixth aspect, which is the process of any one of the first through the fifth aspects, wherein the OCM reaction is characterized by a reaction temperature of from about 750° C. to about 1,000° C.

A seventh aspect, which is the process of any one of the first through the sixth aspects, wherein the catalyst comprises one or more oxides, wherein the one or more oxides comprises CeO₂, La₂O₃—CeO₂, Ca/CeO₂, Mn/Na₂WO₄, Li₂O, Na₂O, Cs₂O, WO₃, Mn₃O₄, CaO, MgO, SrO, BaO, CaO—MgO, CaO—BaO, Li/MgO, MnO, W₂O₃, SnO₂, Yb₂O₃, Sm₂O₃, MnO—W₂O₃, MnO—W₂O₃—Na₂O, MnO—W₂O₃—Li₂O, SrO/La₂O₃, La₂O₃, Ce₂O₃, La/MgO, La₂O₃—CeO₂—Na₂O, La₂O₃—CeO₂—CaO, Na₂O—MnO—WO₃—La₂O₃, La₂O₃—CeO₂—MnO—WO₃—SrO, Na—Mn—La₂O₃/Al₂O₃, Na—Mn—O/SiO₂, Na₂WO₄—Mn/SiO₂, Na₂WO₄—Mn—O/SiO₂, Na/Mn/O, Na₂WO₄, Mn₂O₃/Na₂WO₄, Mn₃O₄/Na₂WO₄, MnWO₄/Na₂WO₄, MnWO₄/Na₂WO₄, Mn/WO₄, Na₂WO₄/Mn, Sr/Mn—Na₂WO₄, or combinations thereof.

An eighth aspect, which is the process of any one of the first through the seventh aspects, wherein an oxygen conversion is from about 90% to 100%.

A ninth aspect, which is the process of any one of the first through the eighth aspects, wherein the byproducts comprise carbon monoxide, carbon dioxide, water, and hydrogen.

A tenth aspect, which is the process of any one of the first through the ninth aspects, wherein producing olefins is a multi-stage process, wherein a first stage comprises steps (a) through (c), and wherein the multi-stage process further comprises one or more additional stages downstream of the first stage, as necessary to achieve a target productivity and/or a target C₂₊ selectivity for the overall multi-stage process.

An eleventh aspect, which is the process of the tenth aspect, wherein each additional stage comprises (i) introducing a reactant mixture to a reactor, wherein the reactant mixture comprises methane, oxygen, and water, wherein the reactor comprises a catalyst, and wherein the water is present in the reactant mixture in an amount of from about 0.5 mol % to about 20 mol %; (ii) allowing at least a portion of the reactant mixture to contact the catalyst and react via an OCM reaction to form a product mixture, wherein the product mixture comprises C₂₊ hydrocarbons, unreacted methane, and byproducts, and wherein the C₂₊ hydrocarbons comprise olefins and paraffins; and (iii) recovering at least a portion of the product mixture from the reactor.

A twelfth aspect, which is the process of the eleventh aspect, wherein the reactant mixture comprises a portion of an upstream product mixture recovered from an upstream reactor.

A thirteenth aspect, which is the process of the twelfth aspect further comprising (i) removing a portion of water from the upstream product mixture to produce an intermediate mixture; and (ii) contacting at least a portion of the intermediate mixture with oxygen to produce the reactant mixture, wherein the reactant mixture comprises water in an amount of from about 0.5 mol % to about 20 mol %.

A fourteenth aspect, which is the process of any one of the first through the thirteenth aspects, wherein the reactant mixture comprises a portion of a downstream product mixture recovered from a downstream reactor.

A fifteenth aspect, which is the process of any one of the first through the fourteenth aspects, wherein the multi-stage process has from 2 to about 5 stages.

A sixteenth aspect, which is a process for producing olefins comprising (a) introducing a first reactant mixture to a first reactor, wherein the first reactant mixture comprises methane, oxygen, and water, wherein the first reactor comprises a first catalyst, and wherein the water is present in the first reactant mixture in an amount of from about 0.5 mol % to about 20 mol %; (b) allowing at least a portion of the first reactant mixture to contact the first catalyst and react via an oxidative coupling of methane (OCM) reaction to form a first product mixture; wherein the first product mixture comprises C₂₊ hydrocarbons, unreacted methane, and byproducts; wherein the C₂₊ hydrocarbons comprise olefins and paraffins; and wherein the byproducts comprise carbon monoxide, carbon dioxide, water, and hydrogen; (c) recovering at least a portion of the first product mixture from the first reactor; (d) removing a portion of the water from the first product mixture to produce a first intermediate mixture; (e) introducing a second reactant mixture to a second reactor comprising a second catalyst, wherein the second reactant mixture comprises at least a portion of the first intermediate mixture and oxygen, wherein the second reactant mixture comprises water in an amount of from about 0.5 mol % to about 20 mol %, and wherein the first catalyst and the second catalyst are the same or different; (f) allowing at least a portion of the second reactant mixture to contact the second catalyst and react via an OCM reaction to form a second product mixture; wherein the second product mixture comprises C₂₊ hydrocarbons, unreacted methane, and byproducts; wherein an amount of unreacted methane in the second product mixture is less than an amount of unreacted methane in the first product mixture; and wherein an amount of olefins in the second product mixture is greater than an amount of olefins in the first product mixture; (g) recovering at least a portion of the second product mixture from the second reactor; (h) optionally removing a portion of the water from the second product mixture to produce a second intermediate mixture; and (i) recovering at least a portion of the olefins from the second product mixture and/or the second intermediate mixture.

A seventeenth aspect, which is the process of the sixteenth aspect, wherein the process is characterized by an overall productivity, an overall C₂₊ selectivity, or both that is increased when compared to an overall productivity, an overall C₂₊ selectivity, or both, respectively, of an otherwise similar process conducted with (i) a first reactant mixture comprising methane and oxygen without the water present in the first reactant mixture in an amount of from about 0.5 mol % to about 20 mol %, and (ii) a second reactant mixture comprising methane and oxygen without the water present in the second reactant mixture in an amount of from about 0.5 mol % to about 20 mol %.

An eighteenth aspect, which is the process of any one of the sixteenth and the seventeenth aspects, wherein producing olefins is a multi-stage process, wherein a first stage comprises steps (a) through (d), wherein a second stage comprises steps (e) through (h), and wherein the multi-stage process further comprises one or more additional stages downstream of the first stage and/or the second stage, as necessary to achieve a target productivity and/or a target C₂₊ selectivity for the overall multi-stage process.

A nineteenth aspect, which is the process of the eighteenth aspect, wherein each additional stage comprises (i) introducing a reactant mixture to a reactor, wherein the reactant mixture comprises methane, oxygen, and water, wherein the reactor comprises a catalyst, and wherein the water is present in the reactant mixture in an amount of from about 0.5 mol % to about 20 mol %; (ii) allowing at least a portion of the reactant mixture to contact the catalyst and react via an OCM reaction to form a product mixture, wherein the product mixture comprises C₂₊ hydrocarbons, unreacted methane, and byproducts, wherein the C₂₊ hydrocarbons comprise olefins and paraffins, and wherein the byproducts comprise carbon monoxide, carbon dioxide, water, and hydrogen; and (iii) recovering at least a portion of the product mixture from the reactor; and (iv) optionally removing a portion of the water from the product mixture to produce an intermediate mixture.

A twentieth aspect, which is the process of the nineteenth aspect, wherein the reactant mixture comprises at least a portion of an upstream intermediate mixture recovered from an upstream reactor.

A twenty-first aspect, which is the process of any one of the sixteenth through the twentieth aspects, wherein the multi-stage process has from 3 to about 5 stages.

A twenty-second aspect, which is a system for producing olefins comprising (a) a first oxidative coupling of methane (OCM) stage comprising: (i) a first adiabatic reactor comprising a first catalyst, wherein the first adiabatic reactor is configured to receive a first reactant mixture comprising methane, oxygen, and water, wherein the water is present in the first reactant mixture in an amount of from about 0.5 mol % to about 20 mol %; and to produce a first product mixture; wherein the first product mixture comprises C₂₊ hydrocarbons, unreacted methane, and byproducts; wherein the C₂₊ hydrocarbons comprise olefins and paraffins; and wherein the byproducts comprise carbon monoxide, carbon dioxide, water, and hydrogen; and (ii) a first separating unit configured to receive at least a portion of the first product mixture and to produce a first intermediate mixture, wherein an amount of water in the first intermediate mixture is less than an amount of water in the first product mixture; (b) a second OCM stage comprising: (iii) a second adiabatic reactor comprising a second catalyst, wherein the second adiabatic reactor is configured to receive a second reactant mixture comprising at least a portion of the first intermediate mixture and oxygen, wherein the water is present in the second reactant mixture in an amount of from about 0.5 mol % to about 20 mol %; and to produce a second product mixture; wherein the second product mixture comprises C₂₊ hydrocarbons, unreacted methane, and byproducts; wherein an amount of unreacted methane in the second product mixture is less than an amount of unreacted methane in the first product mixture; and wherein an amount of olefins in the second product mixture is greater than an amount of olefins in the first product mixture; and (iv) an optional second separating unit configured to receive at least a portion of the second product mixture and to produce a second intermediate mixture, wherein an amount of water in the second intermediate mixture is less than an amount of water in the second product mixture; and (c) a third separating unit configured to receive at least a portion of the second product mixture and/or the second intermediate mixture and to produce olefins.

A twenty-third aspect, which is the system of the twenty-second aspect, wherein the system is characterized by an overall productivity, an overall C₂₊ selectivity, or both that is increased when compared to an overall productivity, an overall C₂₊ selectivity, or both, respectively, of an otherwise similar system having (i) a first reactant mixture comprising methane and oxygen without the water present in the first reactant mixture in an amount of from about 0.5 mol % to about 20 mol %, and (ii) a second reactant mixture comprising methane and oxygen without the water present in the second reactant mixture in an amount of from about 0.5 mol % to about 20 mol %.

A twenty-fourth aspect, which is the system of any one of the twenty-second and the twenty-third aspects, wherein the water is present in the first reactant mixture and in the second reactant mixture in the form of steam in the first adiabatic reactor and the second adiabatic reactor, respectively.

While aspects of the disclosure have been shown and described, modifications thereof can be made without departing from the spirit and teachings of the invention. The aspects and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an aspect of the present invention. Thus, the claims are a further description and are an addition to the detailed description of the present invention. The disclosures of all patents, patent applications, and publications cited herein are hereby incorporated by reference.

1. A process for producing olefins comprising:
(a) introducing a reactant mixture to a reactor, wherein the reactant mixture comprises methane, oxygen, and water, wherein the reactor comprises a catalyst, and wherein the water is present in the reactant mixture in an amount of from about 0.5 mot % to about 20 mol %;
(b) allowing at least a portion of the reactant mixture to contact the catalyst and react via an oxidative coupling of methane (OCM) reaction to form a product mixture; wherein the product mixture comprises C₂₊ hydrocarbons, unreacted methane, and byproducts; Wherein the C₂₊ hydrocarbons comprise olefins and paraffins; and wherein the process is characterized by a productivity, a C₂₊ selectivity, or both that is increased when compared to a productivity, a C₂₊ selectivity, or both, respectively, of an otherwise similar process conducted (i) with a reactant mixture comprising methane and oxygen and (ii) without the water present in the reactant mixture in an amount of from about 0.5 mol % to about 20 mol %;
(c) recovering at least a portion of the product mixture from the reactor;
(d) recovering at least a portion of the C₂₊ hydrocarbons from the product mixture; and
(e) recovering at least a portion of the olefins from the C₂₊ hydrocarbons.

2. The process of claim 1, wherein the reactor is an adiabatic reactor.

3. The process of wherein the process is characterized by a productivity that is increased by equal to or greater than about 1% when compared to a productivity of an otherwise similar process conducted (i) with a reactant mixture comprising methane and oxygen and (ii) without the water present in the reactant mixture in an amount of from about 0.5 mol % to about 20 mol %.

4. The process of claim 1, wherein the process is characterized by a C₂₊ selectivity that is increased by equal to or greater than about 1% when compared to a C₂₊ selectivity of an otherwise similar process conducted (i) with a reactant mixture comprising methane and oxygen and (ii) without the water present in the reactant mixture in an amount of from about 0.5 mol % to about 20 mol %.

5. The process of claim 1, wherein the process is characterized by a productivity that is increased by equal to or greater than about 2% when compared to a productivity of an otherwise similar process conducted (i) with a reactant mixture comprising methane and oxygen and (ii) without the water present in the reactant mixture in an amount of from about 0.5 mol % to about 20 mol %; and wherein the process is characterized by a C₂₊ selectivity that is increased by equal to or greater than about 1% when compared to a C₂₊ selectivity of an otherwise similar process conducted (i) with a reactant mixture comprising methane and oxygen and (ii) without the water present in the reactant mixture in an amount of from about 0.5 mol % to about 20 mol %.

6. The process of claim 1, wherein the OCM reaction is characterized by a reaction temperature of from about 750° C. to about 1,000° C.

7. The process of claim 1, wherein the catalyst comprises one or more oxides, wherein the one or more oxides comprises CeO₂, La₂O₃—CeO₂, Ca/CeO₂, Mn/Na₂WO₄, Li₂O, Na₂O, Cs₂O, WO₃, Mn₃O₄, CaO, MgO, SrO, BaO, CaO—MgO, CaO—BaO, Li/MgO, MnO, W₂O₃, SnO₂, Yb₂O₃, Sm₂O₃, MnO—W₂O₃, MnO—W₂O₃—Na₂O, MnO—W₂O₃—Li₂O, SrO/La₂O₃, La₂O₃, Ce₂O₃, La/MgO, La₂O₃—CeO₂—CaO, MnO—WO₃—La₂O₃, La₂O₃—CeO₂—MnO—WO₃—SrO, Na—Mn—La₂O₃/Al₂O₃, Na—Mn—O/SiO₂, Na₂WO₄—Mn/SiO₂, Na₂WO₄—Mn—O/SiO₂, Na/Mn/O, Na₂WO₄, Mn₂O₃/Na₂WO₄, MnO₄/Na₂WO₄, MnWO₄/Na₂WO₄, MnWO₄/Na₂WO₄, Mn/WO₄, Na₂WO₄/Mn, Sr/Mn—Na₂WO₄, or combinations thereof.

8. The process of claim 1, wherein producing olefins is a multi-stage process, wherein a first stage comprises steps (a) through (c), and wherein the multi-stage process further comprises one or more additional stages downstream of the first stage, as necessary to achieve a target productivity and/or a target C₂₊ selectivity for the overall multi-stage process.

9. The process of claim 8, wherein each additional stage comprises (i) introducing a reactant mixture to a reactor, wherein the reactant mixture comprises methane, oxygen, and water, wherein the reactor comprises a catalyst, and wherein the water is present in the reactant mixture in an amount of from about 0.5 mol % to about 20 mol %; (ii) allowing at least a portion of the reactant mixture to contact the catalyst and react via an OCM reaction to form a product mixture, wherein the product mixture comprises C₂₊ hydrocarbons, unreacted methane, and byproducts, and wherein the C₂₊ hydrocarbons comprise olefins and paraffins; and (iii) recovering at least a portion of the product mixture from the reactor.

10. The process of claim 9, wherein the reactant mixture comprises a portion of an upstream product mixture recovered from an upstream reactor.

11. The process of claim 10 further comprising (i) removing a portion of water from the upstream product mixture to produce an intermediate mixture; and (ii) contacting at Least a portion of the intermediate mixture with oxygen to produce the reactant mixture, wherein the reactant mixture comprises water in an amount of from about 0.5 mol % to about 20 mol %.

12. The process of claim 9, wherein the reactant mixture Comprises a portion of a downstream product mixture recovered from a downstream reactor.

13. The process of claim 8, wherein the multi-stage process has from 2 to about 5 stages.

14. A process for producing olefins comprising:
(a) introducing a first reactant mixture to a first reactor, wherein the first reactant mixture comprises methane, oxygen, and water, wherein the first reactor comprises a first catalyst, and wherein the water is present in the first reactant mixture in an amount of from about 0.5 mol % to about 20 mol %;
(b) allowing at least a portion of the first reactant mixture to contact the first catalyst and react via an oxidative coupling of ethane (OCM) reaction to form a first product mixture; wherein the first product mixture comprises C₂₊ hydrocarbons, unreacted methane, and byproducts; wherein the C₂₊ hydrocarbons comprise olefins and paraffins; and wherein the byproducts comprise carbon monoxide, carbon dioxide, water, and hydrogen;
(c) recovering at least a portion of the first product mixture from the first reactor;
(d) removing a portion of the water from the first product mixture to produce a first intermediate mixture;
(e) introducing a second reactant mixture to a second reactor comprising a second catalyst, wherein the second reactant mixture comprises at least a portion of the first intermediate mixture and oxygen, wherein the second reactant mixture comprises water in an amount of from about 0.5 mol % to about 20 mol %, and wherein the first catalyst and the second catalyst are the same or different;
(f) allowing at least a portion of the second reactant mixture to contact the second catalyst and react via an OCM reaction to form a second product mixture; wherein the second product mixture comprises C₂₊ hydrocarbons, unreacted methane, and byproducts; wherein an amount of unreacted methane in the second product mixture is less than an amount of unreacted methane in the first product mixture; and wherein an amount of olefins in the second product mixture is greater than an amount of olefins in the first product mixture;
(g) recovering at least a portion of the second product mixture from the second reactor;
(h) optionally removing a portion of the water from the second product mixture to produce a second intermediate mixture; and
(i) recovering at least a portion of the olefins from the second product mixture and/or the second intermediate mixture.

15. The process of claim 14, wherein the process is characterized by an overall productivity, an overall C₂₊ selectivity, or both that is increased when compared to an overall productivity, an overall C₂₊ selectivity, or both, respectively, of an otherwise similar process conducted with (i) a first reactant mixture comprising methane and oxygen without the water present in the first reactant mixture in an amount of from about 0.5 mol % to about 20 mol %, and (ii) a second reactant mixture comprising methane and oxygen without the water present in the second reactant mixture in an amount of from about 0.5 mol % to about 20 mol %.

16. The process of claim 14, wherein producing olefins is a multi-stage process, wherein a first stage comprises steps (a) through (d), wherein a second stage comprises steps (e) through (h), and wherein the multi-stage process further comprises one or more additional stages downstream of the first stage and/or the second stage, as necessary to achieve a target productivity and/or a target C₂₊ selectivity for the overall multi-stage process.

17. The method of claim 16, wherein each additional stage comprises (i) introducing a reactant mixture to a reactor, wherein the reactant mixture comprises methane, oxygen, and water, wherein the reactor comprises a catalyst, and wherein the water is present in the reactant mixture in an amount of from about 0.5 mol % to about 20 mol %; (ii) allowing at least a portion of the reactant mixture to contact the catalyst and react via an OCM reaction to form a product mixture, wherein the product mixture comprises C hydrocarbons, unreacted methane, and byproducts, wherein the C₂₊ hydrocarbons comprise olefins and paraffins, and wherein the byproducts comprise carbon monoxide, carbon dioxide, water, and hydrogen; and (iii) recovering at least a portion of the product mixture from the reactor; and (iv) optionally removing a portion of the water from the product mixture to produce an intermediate mixture.

18. The process of claim 17, wherein the reactant mixture comprises at least a portion of an upstream intermediate mixture recovered from an upstream reactor.

19. The method of claim 16, wherein the multi-stage process has from 3 to about 5 stages.

20. A system for producing olefins comprising:
(a) a first oxidative coupling of methane (OCM) stage comprising:
(i) a first adiabatic reactor comprising a first catalyst, wherein the first adiabatic reactor is configured to receive a first reactant mixture comprising methane, oxygen, and water, wherein the water is present in the first reactant mixture in an amount of from about 0.5 mol % to about 20 mol %; and to produce a first product mixture; wherein the first product mixture comprises C₂₊ hydrocarbons, unreacted methane, and byproducts; wherein the C₂₊ hydrocarbons comprise olefins and paraffins; and wherein the byproducts comprise carbon monoxide, carbon dioxide, water, and hydrogen; and
(ii) a first separating unit configured to receive at least a portion of the first product mixture and to produce a first intermediate mixture, wherein an amount of water in the first intermediate mixture is less than an amount of water in the first product mixture;
(b) a second OCM stage comprising:
(iii) a second adiabatic reactor comprising a second catalyst, wherein the second adiabatic reactor is configured to receive a second reactant mixture comprising at least a portion of the first intermediate mixture and oxygen, wherein the water is present in the second reactant mixture in an amount of from about 0.5 mol % to about 20 mol %; and to produce a second product mixture; wherein the second product mixture comprises C₂₊ hydrocarbons, unreacted methane, and byproducts; wherein an amount of unreacted methane in the second product mixture is less than an amount of unreacted methane in the first product mixture; and wherein an amount of olefins in the second product mixture is greater than an amount of olefins in the first product mixture; and
(iv) an optional second separating unit configured to receive at least a portion of the second product mixture and to produce a second intermediate mixture, wherein an amount of water in the second intermediate mixture is less than an amount of water in the second product mixture; and
(c) a third separating unit configured to receive at least a portion of the second product mixture and/or the second intermediate mixture and to produce olefins.
